(19) United States
(12) Patent Application Publication
(10) Pub. No.: US 2019/0194513 A1
(43) Pub. Date: Jun. 27, 2019

(54) USE OF AQUEOUS SOLUTION OF ORGANIC AMMONIUM CARBOXYLATE IN PREVENTING DUSTING OF FINE MATERIAL AND COMBINATION OF AN AQUEOUS SOLUTION OF ORGANIC AMMONIUM CARBOXYLATE AND FINE MATERIAL

(71) Applicant: Oy Granula Ab Ltd., Kotka (FI)

(72) Inventor: Thomas AHLNÄS, Kotka (FI)

(73) Assignee: Oy Granula Ab Ltd., Kotka (FI)

(21) Appl. No.: 15/852,432

(22) Filed: Dec. 22, 2017

(51) Int. Cl. C09K 3/22; C09K 3/18; C07C 211/63; B28D 7/02; E21F 5/02

(52) U.S. Cl. CPC C09K 3/22; C09K 3/18; E21F 5/02; B28D 7/02; C07C 211/63

(57) ABSTRACT

A method to control dusting of sand, crushed stone, mineral dust, metal powder and the like by using an aqueous solution of organic of ammonium carboxylate is disclosed.

### FIELD OF INVENTION

The invention relates to use of an aqueous solution of organic ammonium carboxylate in controlling dusting of sand, crushed stone, mineral dust, metal powder and the like. The invention also relates to combination of an aqueous solution of organic ammonium carboxylate and fine material. The organic ammonium carboxylate which is used as an aqueous solution is especially adapted for controlling dust formation in applications where the biodegradation and low BOD is also required. Preferably the present invention relates to the use of environmentally benign freezing point depressant compositions for preventing dust formation and for preventing ice formation (anti-icing) within the compositions itself.

### BACKGROUND OF INVENTION

Mineral dust and street dust are serious health problems for example in mining industry and cities. There are also other applications wherein dusting of sand, fine crushed stone and soil will cause mineral dust which is a serious health problem. Dusting causes also damage to equipment and vehicle used for example in mining industry, these include vehicles, electric motors, transport bands etc. Specifically, in mining industry mineral, dust will intrude itself into ore silos and transportation vehicles and in mining equipment causing freezing in winter time.

Several ways to reduce mineral dust in mining industry have been suggested. One alternative is to bind mineral dust by means of aqueous or water-borne solutions. However, none of water-borne solutions have been successful this far.

If an aqueous solution is used in dust control, water has a tendency to evaporate after been sprayed onto surface of crushed stone, sand or soil. This requires usage of relatively large amounts of aqueous solution. Using plenty of water in binding dust will then cause other problems, such as mineral puddling.

One important aspect when using aqueous solutions in controlling mineral dust is possible disturbances brought into mining industry processes alongside with said aqueous solution: especially chlorides of potassium, magnesium, calcium and sodium have a tendency to cause disturbances in ore recovery processes, for example in extraction stages.

One important aspect is also biodegradability of solutions used for controlling mineral dust.

Additionally, using aqueous solutions to dust binding may also cause freezing of aqueous solution itself. Freezing point depressant compositions are used widespread for variety of purposes, especially for lowering freezing point of an aqueous system so that ice cannot be formed on surfaces or within the aqueous systems or for melting ice formed in those aqueous systems. However, dust control with freezing point depressant compositions are relative rare because the effectiveness of the freezing point depressant compositions depends on the molar freezing point lowering effect, the number of ionic species that are made available, and to the degree to which the compositions can be dispersed in the liquid phase.

Most freezing point depressant compositions are either based on salts, such as sodium chloride or potassium formate, or alcohols such as glycols and methanol. Alkali and alkali-earth metal salts of carboxylic acid such as potassium formate, sodium formate, potassium acetate and sodium acetate have found increasing use in the area of deicing mainly due to their low environment impact and low viscosity at minus temperatures. However, using these compositions for controlling dusting in mining industry is not a viable option, because potassium, natrium, magnesium and calcium chlorides may interfere ore recovering process.

### GENERAL DESCRIPTION OF THE INVENTION

The above back ground as a starting point, the object of the present invention was to solve or at least to alleviate above mentioned problems.

Thus, the general object of the present invention was to provide an aqueous solution which could be used as a combined freezing point depressant and an aqueous solution for controlling dusting and which is also environmentally benign, and which do not form chlorides which may interfere mining process.

The ideal dusting control agent and freezing point depressant composition adapted to use for mining industry would have following properties:

it should prevent effectively formation of mineral dust from grounded stones, sand and soil,

it should be free of mining process disturbing halides such as chlorides of alkali and alkali-earth metals especially chlorides of potassium, sodium, magnesium and calcium,

it should have relatively low biological (BOD) and chemical oxygen demand (COD),

it should be effective at low temperatures, i.e. it should have low viscosity and low freezing point,

it should not cause mineral puddling, that is, it should be effective when used also as relatively small amounts.

The present inventors have surprisingly found that the ideal solution for above mentioned problems relating to reducing mineral dust in air with an aqueous solution and in the same time lowering the freezing point of said aqueous solution itself is to use specific aqueous solution of organic ammonium carboxylate of formula (I):

[NR¹R²R³R⁴]⁺ₙ [R⁵(COO)]⁻ⁿ, (I),

in which R¹, R², and R³ are selected from the group composing of hydrogen and methyl, R⁴ is a C₁-C₄-alkyl substituted with a hydroxyl group, R⁵ is hydrogen or methyl and n is 1,

as a mist, in preventing dusting of fine material selected from the group composing of sand, crushed stone, minerals and metal powder.

Organic ammonium carboxylate stands for a salt or a complex formed of an ammonium cation and a carboxylic anion. Hence one or more ammonium ions of the salt or complex may be primary (RNH₃⁺), secondary (R₂NH₂⁺), tertiary (R₃NH⁺) or quaternary (R₄N⁺). The carboxylate ion of the salt or complex may be monovalent (RCOO⁻) or polyvalent (R(COO⁻)_{n>1}), and in that case it may also comprise unneutralised carboxyl groups (—COOH). In the latter case, R⁵ is defined as being substituted with carboxyl.

Since the group R⁵ is associated with a carboxylate group of formic acid or acetic acid, the ammonium carboxylate of formula (I) is based on formic acid or acetic acid and it can be prepared from such an acid or its salt.

In formula (I), n is 1. Consequently, organic ammonium carboxylates used for controlling mineral dust are based on lower fatty acids.

As mentioned above, the ammonium ion of formula (I) may be primary (RNH₃⁺), secondary (R₂NH₂⁺), tertiary (R₃NH⁺) or quaternary (R₄N. Typical ammonium ions containing unsubstituted alkyls have been formed from water-soluble amines such as methylamine (g), dimethylamine, trimethylamine, ethylamine, diethylamine, etc.

Ammonium ions containing substituted alkyls have typically been formed from water-soluble amines, whose alkyl(s) may have been substituted with one or more hydroxyl groups. In formula (1), R₁ is preferably hydrogen and R₂ and R₃ have been selected from the group comprising hydrogen and methyl. R₄ is C₁-C₄-alkyl substituted with a hydroxyl group.

Organic ammonium carboxylates formed of lower alkanolamines are hence particularly useful. Among lower alkanolamines we may cite monoethanolamine. Preferable aqueous solutions of ammonium carboxylates of formula (I) contain formic acid, acetic acid and monoethanolamine or trimethylmonoethanolamine. Trimethylmonoethanolamine is also called as acetylcholine.

One important group of useful alkanolamines comprises lower alkyl alkanolamines, such as methyl ethanolamine, dimethylethanolamine. Additional information about useful alkanolamines can be found in the book Kirk-Othmer, Encyclopedia of Chemical Technology 3rd Ed., Vol. 1, p. 944, which is incorporated in this disclosure.

It is particularly recommendable that R₁ is hydrogen, R₂ and R₃ are selected from the group comprising of hydrogen and methyl and R₄ is ethyl substituted with a hydroxyl group, preferably 2-hydroxy ethyl. In the most advantageous embodiment, the organic ammonium carboxylate of formula (I) is selected from the group comprising of a salt or a complex of formic acid or acetic acid and monoethanolamine or trimethylethanolamine.

In the practice, ammonium carboxylate of formula (I) is prepared e.g. by mixing an ammonium cation source and a carboxyl anion source in the desired molar ratio, either without a medium or by using an appropriate solvent such as water as a medium. When the starting materials are an amine and an acid, they are simply mixed during gentle heating, if necessary. When the starting materials consist of salts, they are typically dissolved separately in water, and then the solutions are combined. If a salt or a complex thus formed is hydrophobic, it will separate from the water phase as an unctuous or paste-like deposit or a wax-like precipitate, and it can be separated from the water phase by any known methods. When both the starting materials and the formed product are hydrophobic, the preparation can be carried out in an organic solvent instead of water. The freezing point depressant composition used in the invention comprises either fluid composing of ammonium carboxylate of formula (I) without solvent or ammonium carboxylate of formula (I) with appropriate solvent. Preferably solvent is an aqueous solution or a dispersion. Chemical stability: Preliminary results indicate that for instance a fluid pair: ethylene amide—formic acid could under special circumstances react and form amid when no solvent is present. Increasing the temperature favours amid formation. Nearly no esters are formed.

To be exact the invention relates the use defined in claim 1 and also a combination defined in claim 14 comprising of droplets of aqueous solution of organic ammonium carboxylate and fine material.

In the use according to present invention aqueous solution of organic ammonium carboxylate of formula (I):

[NR¹R²R³R⁴]⁺ₙ [R⁵(COO)]⁻ⁿ, (I),

in which R¹, R², and R³ are selected from the group composing of hydrogen and methyl, R⁴ is a C₁-C₄-alkyl substituted with a hydroxyl group, R⁵ is hydrogen or methyl and n is 1,

is used as a mist, in preventing dusting of fine material selected from the group composing of sand, crushed stone, minerals and metal powder and in lowering the freezing point of said aqueous solution.

In the combination according to present invention there exists droplets of aqueous solution of organic ammonium carboxylate of formula (I)):

[NR¹R²R³R⁴]⁺ₙ [R⁵(COO)]⁻ⁿ, (I),

in which R¹, R², and R³ are selected from the group composing of hydrogen and methyl, R⁴ is a C₁-C₄-alkyl substituted with a hydroxyl group, R⁵ is hydrogen or methyl and n is 1,

and fine material is selected from the group composing of sand, crushed stone, minerals and metal powder and

wherein said aqueous solution of organic ammonium carboxylate of formula (I) have been sprayed as a mist onto the surface of said fine material.

In one preferably use, aqueous solution of organic ammonium carboxylate of formula (I) is used as a mist 1.0-2.0 I per 1000 kg of crushed stone, preferably 1.2-1.5 I per 1000 kg of crushed stone.

The aqueous solution of organic ammonium carboxylate (3-5% solution) have a freezing point from −3° C. to −15° C. preferably—from 5 to −10° C. However, after applied onto surface of fine material (for example crushed stone) the water will evaporate from said aqueous solution. This will automatically lower the freezing point of remaining water in said combination of water and organic ammonium carboxylate: as can be seen later from (table IV) freezing point of aqueous solution of organic ammonium carboxylate of formula (I) will be considerably lowered when water leaves and rest of fluid comes more concentrated.

The aqueous solution of organic ammonium carboxylate is compatible with ammonium salts of C₁-C₆ monocarboxylic acids or carboxylates of urea or ethylene glycol or propylene glycol, or glycerol or a mixture thereof. We include in the invention the use of combinations of the ammonium carboxylate of formula (I) with an ammonium salts of C₁-C₆ monocarboxylic acids or urea or ethylene glycol or propylene glycol, or glycerol or a mixture thereof carboxylates. By using aqueous solution of organic ammonium carboxylate with freezing point depressant composition with urea for de-icing or anti icing applications one can lower oxygen demand on the environment.

The aqueous solution of organic ammonium carboxylate of formula (I) may contain auxiliary substances as well. Typical auxiliary substances comprise such as additional corrosion inhibitors, biocides, coloring agents, surfactants, and viscosity intensifiers. The concentration of auxiliary substances will be in the range of 0.001 to 10 wt-% from the total weight of aqueous solution.

In a preferred embodiment of the invention the aqueous solution of organic ammonium carboxylate of formula (I) have concentration of sodium, potassium, calcium and magnesium 0 M. This means that there will be no chlorides formation from these cations after said aqueous solution have been applied for controlling dusting in mines.

Once applied onto surface of above defined fine material, drying of said combination will lead on the one hand to water loss and on the other hand absorbing of water by way of the organic ammonium carboxylate of formula (I) resulting in keeping the surface of said fine material moist. Actually organic ammonium carboxylate of formula (I) will absorb water after water content of said aqueous solution have dropped at or below 15 wt-%. This is a very important aspect of the present invention, because it will reduce drastically the amount of aqueous solution of organic ammonium carboxylate of formula (I) needed to keep dusting in control after applied onto fine material surface.

Biodegradability of aqueous solutions of the invention used for controlling dusting is relatively low: droplets of aqueous solution of organic ammonium carboxylate of formula (I), and fine material, wherein carboxylate originate from acetic acid, in form of fluid have BOD (biological oxygen demand) of 0.64 mg of O₂ at 20° C. for liter of said fluid in 5 days. COD (chemical oxygen demand) for the same combination 0.64 mg O₂ at 20° C. for liter of said fluid in 5 days.

### SHORT DESCRIPTION OF THE FIGURES

FIG. 1 shows the freezing points of aqueous solutions of organic ammonium carboxylate of formula (I), wherein R⁵=H (formiate).

### DESCRIPTION OF PREFERRED EMBODIMENTS OF THE INVENTION

The invention is described below in more greater details with the help of examples. Person skilled in the art will recognize that the properties of the compositions studied are such that they will make ideal mineral dust control aqueous solutions having also freezing point depressant properties for binding dusting of streets, ore mines, horse tracks etc.

In the following non-restricting examples we have presented some specific applications and properties of aqueous solutions comprising organic ammonium carboxylate of formula (I) as well as combinations comprising aqueous solution of organic ammonium carboxylate of formula (I) and fine material selected from the group composing of sand, crushed stone, minerals and metal powder.

### EXAMPLE 1

An ionogenic solution for controlling mineral dust formation was prepared by mixing 1 mole of formic acid (99%) with 1 mole of monoethanolamine (99%). Distilled water was added to the fluid mixture in order to made 3-5% by weight aqueous solution.

The freezing point of the solution was below −5° C., the electrical conductivity of the fluid was 61 mS/cm at 26° C., and pH of the fluid was 7.55 (measured directly from the solution).

### EXAMPLE 2

An aqueous solution was prepared by mixing 1 mole of formic acid (99%) with 1 mole of monoethanolamine (99%). Distilled water was added to the fluid mixture in order to made 3-5 by weight solution in water.

The freezing point of the solution was below −5° C., the Brookfield DV-I viscosity (20 rpm) was 10 mPas at −20° C., 10 mPas at −10° C., 10 mPas at 0° C., and Bohlin VOR viscosity (shear rate 23.1 1/s) was 4 mPas at 10° C., 3 mPas at 20° C., 2 mPas at 40° C., and 1.5 mPas at 60° C. The electrical conductivity of the fluid was 65 mS/cm at 26° C., and pH of the fluid was 7.54 (measured directly from the solution).

### EXAMPLE 3

Solutions in examples 6 and 7 in the below tables have been made in the same way as presented in examples 1-2, that is, by mixing 1 mole of an ammonium cation source and 1 mole of a carboxyl anion source (unless otherwise shown) together for obtaining a concentrated fluid and then adding water to the concentrated fluid, for obtaining diluted solutions.

TABLE 1
In table 1 formation of possible precipitates from fluids and diluted
solutions obtained from fluids is shown. Temperature was 20-25° C.

| | | fluid Wt-% from solution | | | | | | | pH of 2% |
|---|---|---|---|---|---|---|---|---|---|
| Code/ex | fluid | 100 | 90 | 80 | 60 | 40 | 20 | 5 | solution |
| EAE/6 | ethanolamine/ | Clear | Clear | Clear | Clear | Clear | Clear | Clear | 6.8 |
| | acetic acid | | | | | | | | |
| EAM/7 | ethanolamine/ | Clear | Clear | Clear | Clear | Clear | Clear | Clear | 3.7 |
| | formic acid | | | | | | | | |

TABLE 2
The fluid and solution samples from selected examples of table 1 were subjected to chilling
to a temperature of +4° C. and then to further cooling to a temperature of −20° C.
In these temperatures the possible turbidity, precipitation of these samples was observed.

| ex | | 100 | 90 | 80 | 60 | 40 | 20 | 5 |
|---|---|---|---|---|---|---|---|---|
| | Temperature +4 C. | | | | | | | |
| 6 | ethanolamine/ | Clear | Clear | Clear | Clear | Clear | Clear | Clear |
| | acetic acid | | | | | | | |
| 7 | ethanolamine/ | Clear | Clear | Clear | Clear | Clear | Clear | Clear |
| | formic acid | | | | | | | |
| | Temperature −20° C. | | | | | | | |
| 6 | ethanolamine/ | Clear/liquid | Clear/liquid | Clear/liquid | Clear/ | Clear/liquid | frozen | frozen |
| | acetic acid | state | state | state | liquid state | state | | |
| 7 | ethanolamine/ | Clear/ | Clear/ | Clear/liquid | Clear/ | frozen | frozen | frozen |
| | formic acid | liquid | liquid | state | liquid state | | | |
| | | state | state | | | | | |

As can be seen from table 1 and table 2 water based solutions of organic of ammonium carboxylates are clear solutions independent whether they are diluted or not. This means that when they are used in controlling mineral dust they have no tendency to salt out after sprayed onto surface of crushed stones, sand, soil or metal. Therefore they do not interfere for example vehicle brakes or transport belts used in mining processes.

Table 3

In table 3 results from viscosity measurements compositions of examples 6 and 7 are given. Viscosity was measured with Bohlin method (bold numbers) at shear rate 23.1 1/s and with Brookefield method (normal numbers) at shear rate 20 rpm. Additionally, electrical conductivity, pH and redox potential was measured for these compositions comprising fluids and solutions prepared from these fluids by adding distilled water.

TABLE 3

| | | | | | | | | |
|---|---|---|---|---|---|---|---|---|
| monoethanolamine/acetic acid | fluid Wt-% | 100 | 90 | 80 | 60 | 40 | 20 | 5 |
| | from | | | | | | | |
| | solution | | | | | | | |
| water | water wt-% | 0 | 10 | 20 | 40 | 60 | 80 | 95 |
| | ° C. | VISCOSITY | | | Bohlin | | shear rate | |
| | | mPas | | | VOR | | 23.1 1/s | |
| | | | | | viscosity | | 20 rpm sp3 | |
| | | | | | Brookfield | | | |
| | | | | | DV-I | | | |
| | | | | | viscosity | | | |
| viscosity mPas/ | −20 | >20000 | >20000 | 12450 | 170 | 35 | X | X |
| | (repeat) | | | | | | | |
| | −20 | >20000 | 16740 | 1700 | 80 | 20 | X | X |
| | −10 | >20000 | 5150 | 700 | 60 | 15 | 10 | 5 |
| | 0 | 27850 | 2160 | 330 | 40 | 10 | 10 | 5 |
| | 10 | 15250 | 1152 | 210 | 23 | 6 | 2 | 1.7 |
| | 20 | 5665 | 556 | 118 | 15 | 5 | 2 | 1.3 |
| | 40 | 1220 | 154 | 41 | 8 | 3 | 1.5 | 1.1 |
| | 60 | 345 | 63 | 20 | 5 | 2 | 1 | 0.7 |
| conductivity mS/cm | | 0.534 | 2.24 | 7.1 | 25.9 | 46.9 | 47.8 | 20.2 |
| T ° C. | | 25.4 | 25.9 | 26 | 25.6 | 25.4 | 25.1 | 24.9 |
| pH ° C. 22 | | 7.96 | 7.81 | 7.68 | 7.34 | 7.07 | 6.87 | 6.79 |
| REDOX | | +31 | +54 | +69 | +107 | +146 | +179 | +216 |
| | fluid Wt-% | 100 | 90 | 80 | 60 | 40 | 20 | 5 |
| Composition: | from | | | | | | | |
| monoethanolamine/formic acid | solution | | | | | | | |
| water | water wt-% | 0 | 10 | 20 | 40 | 60 | 80 | 95 |
| | pale oily | VISCOSITY | | | Bohlin | | shear rate | |
| | light liquid | mPas | | | VOR | | 23.1 1/s | |
| | ° C. | | | | viscosity | | 20 rpm sp3 | |
| | | | | | Brookfield | | | |
| | | | | | DV-I | | | |
| | | | | | viscosity | | | |
| viscosity mPas/ | −30 | | | | | | | |
| | −20 | 4350 | 680 | 230 | 30 | 10 | X | X |
| | −10 | 2830 | 410 | 130 | 20 | 10 | 5 | X |
| | 0 | 1335 | 240 | 75 | 15 | 10 | 5 | 5 |
| | 10 | 646 | 123 | 41 | 9 | 4 | 2 | 1.5 |
| | 20 | 325 | 72 | 26 | 6 | 3 | 1.7 | 1.2 |
| | 40 | 119 | 31 | 13 | 4 | 2 | 1.2 | 0.95 |
| | 60 | 47 | 17 | 7 | 3 | 1.5 | 1.1 | 0.9 |
| conductivity mS/cm | | 15.9 | 27.3 | 40.4 | 61 | 65 | 46.9 | 16 |
| T ° C. | | 26.1 | 25.9 | 25.8 | 25.6 | 25.5 | 25.5 | 25.8 |
| pH/22° C. | | 7.75 | 7.67 | 7.6 | 7.55 | 7.54 | 7.53 | 7.51 |
| REDOX potential | | −321 | −244 | −164 | −110 | −75 | −48 | +4 |

As can be seen from table 3 the viscosity of compositions varies considerably depending on the quality of the fluid in a composition and fluid—solvent proportion (w/w). No solid crystals will be formed for instance if one uses combination ethanol amine/formic acid. Avoiding solid crystals is also a beneficial property for instance for an aqueous solution used for dust control.

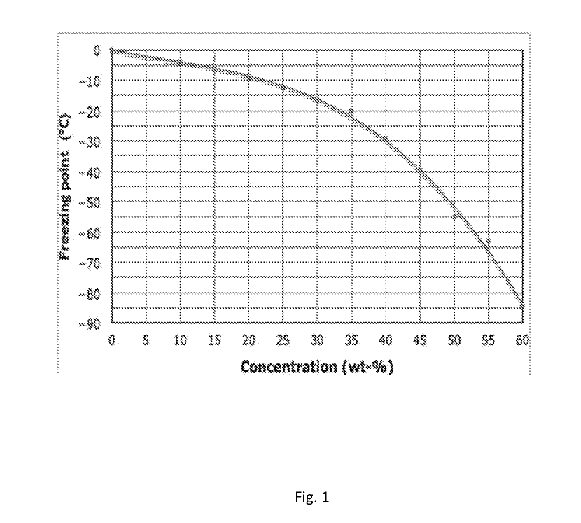

FIG. 1 shows the freezing points of aqueous solutions of organic ammonium carboxylate of formula (I), wherein R⁵=H (formiate). Table 4 below shows the freezing points of selected aqueous solutions of organic ammonium carboxylate of formula (I), wherein R⁵=H (formiate).

TABLE 4
Samples of organic ammonium carboxylate of formula (I)
in water and their freezing points:

| Sample | Concentration (wt-%) | Freezing point (° C.) |
|---|---|---|
| Water | 0 | 0 |
| HTF-20 | 20 | −8.96 |
| HTF-25 | 25 | −12.44 |
| HTF-30 | 30 | −16.51 |
| HTF-35 | 35 | −20.13 |
| HTF-40 | 40 | −29.33 |
| HTF-45 | 45 | −39.48 |
| HTF-50 | 50 | −54.95 |
| HTF-55 | 55 | −63.01 |
| HTF-60 | 60 | −84.50 |

As can be seen from FIG. 1 and table 4: as the concentration of water solutions of organic ammonium carboxylate of formula (I) increases it will readily lead to lower freezing points. For example, those aqueous solutions of organic ammonium carboxylate of formula (I) presented in FIG. 1 and table 4 having concentration about 10 wt-% have a freezing point about −5° C. However, when the concentration of organic ammonium carboxylate of formula (I) in the aqueous solution increases, the freezing point of the aqueous solutions falls considerably, for example when the concentration of aqueous solution is 30 (wt-%) the freezing point of said aqueous solution is about −20° C. When the concentration of aqueous solution is 60 wt-%) the freezing point of said aqueous solution is about −85° C. The freezing point of −5° C. corresponds the freezing point of aqueous solutions of organic ammonium carboxylate of formula (I) which is ready-to-use (1-7 wt % aqueous solution). The freezing point of −30 C and −85% corresponds the freezing point of the aqueous solution of organic ammonium carboxylate of formula (I) which has been sprayed onto surface of fine material when water has been evaporated.

The invention is illustrated by following embodiments:

1. The use of aqueous solution of organic ammonium carboxylate of formula (I):

[NR¹R²R³R⁴]⁺ₙ [R⁵(COO)]⁻ⁿ, (I),

in which R¹, R², and R³ are selected from the group composing of hydrogen and methyl, R⁴ is a C₁-C₄-alkyl substituted with a hydroxyl group, R⁵ is hydrogen or methyl and n is 1,

as a mist, in preventing dusting of fine material selected from the group composing of sand, crushed stone, minerals and metal powder and in lowering the freezing point of said aqueous solution.

2. The use defined in above embodiment, wherein R¹, R² and R³ are all hydrogen and R⁴ is an ethyl substituted with a hydroxyl group, preferably 2-hydroxy ethyl.

3. The use defined in above embodiments, wherein R¹, R² and R³ are all methyl and R⁴ is an ethyl substituted with a hydroxyl group, preferably 2-hydroxy ethyl.

4. The use defined in above embodiments, wherein the organic ammonium carboxylate of formula (I) is a salt of formic acid and monoethanolamine or a salt of acetic acid and monoethanolamine.

5. The use as defined in above embodiments, wherein the concentration of the organic ammonium carboxylate of formula (I) in the aqueous solution, is in the range of 1-7% wt-%, preferably in the range of 3-5 wt-% while the freezing point of the composition is kept in the range of −5 to −50° C.

6. The use as defined in above embodiments, wherein the aqueous solution of organic ammonium carboxylate of formula (I) contains organic ammonium carboxylate of formula (1) and water in a weight ratio in the range of 1:50-1:15, preferably in the range of 1:33-1:20.

7. The use as defined in above embodiments, wherein the aqueous solution of organic ammonium carboxylate of formula (I) contains distilled water only.

8. The use as defined in above embodiments wherein the concentration of sodium, potassium, calcium and magnesium is 0 M.

9. The use as defined in any of above embodiments, wherein aqueous organic ammonium carboxylate of formula (I) is additionally admixed with compounds selected from the group comprising glycols, preferably ethylene glycol or propylene glycol, glycerols and viscosity modifying polymers, so that said aqueous composition contains 5 to 97.5 wt-% of water.

10. The use as defined in above embodiments, wherein aqueous organic ammonium carboxylate of formula (I) is additionally admixed with auxiliary substances such as additional corrosion inhibitors, biocides, coloring agents, surfactants, and viscosity intensifiers, so that the concentration of auxiliary substances will be in the range of 0.001 to 10 wt-% from the total weight of aqueous solution.

11. The use defined in above embodiments, wherein the organic ammonium carboxylate of formula (1) contains formic acid, acetic acid and monoethanolamine or trimethylmonoethanolamine.

12. The use defined in above embodiments, wherein aqueous organic ammonium carboxylate of formula (I) is used 1-2 I per 1000 kg of crushed stone, preferably 1.2-1.5 I per 1000 kg of crushed stone.

13. The use defined in above embodiments, wherein freezing point of said aqueous solution is from −3° C. to −15° C. preferably from −5° C. to −10° C.

14. The use defined in above embodiments, further including a step wherein aqueous organic ammonium carboxylate of formula (I) is prepared by diluting, in situ, concentrated solution of water and organic ammonium carboxylate of formula (I) with water, in which said concentrated solution the concentration of organic ammonium carboxylate of formula (I) is 50 wt-% or 85 wt-%

15. The use defined in above embodiments, further including a step wherein the aqueous organic ammonium carboxylate of formula (I) is prepared by mixing, in situ, an aqueous solution of 1-5 wt-% of amine with an aqueous solution of 1-5 wt-% of acid

16. A combination of droplets of aqueous solution of organic ammonium carboxylate of formula (I):

[NR¹R²R³R⁴]⁺ₙ [R⁵(COO)]⁻ⁿ, (I),

in which R¹, R², and R³ are selected from the group composing of hydrogen and methyl, R⁴ is a C₁-C₄-alkyl substituted with a hydroxyl group, R⁵ is hydrogen or methyl and n is 1,

and fine material selected from the group composing of sand, crushed stone, minerals and metal powder,

wherein said aqueous solution of organic ammonium carboxylate of formula (I) has been sprayed as a mist onto the surface of said fine material.

17. The combination defined in above embodiment comprising of droplets aqueous solution of organic ammonium carboxylate of formula (I) and fine material, wherein drying of said combination will lead on the one hand to water loss and on the other hand absorbing of water by way of the organic ammonium carboxylate of formula (I) resulting in keeping the surface of said fine material moist.

18. The combination defined in above embodiments, comprising droplets of aqueous solution of organic ammonium carboxylate of formula (I) and fine material, wherein organic ammonium carboxylate of formula (I) will absorb water after water content of said aqueous solution have dropped at or below 15 wt-%,

19. The combination defined in above embodiments comprising droplets of aqueous solution of organic ammonium carboxylate of formula (I), and fine material, wherein carboxylate originate from acetic acid, in form of fluid and having BOD (biological oxygen demand) of 0.64 mg of O₂ at 20° C. for liter of said fluid in 5 days.

20. The combination defined in above embodiments comprising droplets of aqueous solution of organic ammonium carboxylate of formula (I), and fine material, wherein carboxylate originate from acetic acid, in form of fluid, and having COD (chemical oxygen demand) of 0.64 mg O₂ at 20° C. for liter of said fluid in 5 days.

What is claimed is:

1. A method to prevent dusting of fine materials, said method comprising the steps of
providing a mist of an aqueous solution of organic ammonium carboxylate of formula (I):
[NR¹R²R³R⁴]⁺ₙ [R⁵(COO)]⁻ⁿ, (I),
in which R¹, R², and R³ are selected from the group composing of hydrogen and methyl, R⁴ is a C₁-C₄-alkyl substituted with a hydroxyl group, R⁵ is hydrogen or methyl and n is 1,
applying the mist on the fine material, wherein the fine material is selected from the group composing of sand, crushed stone, minerals and metal powder.

2. The method of claim 1, wherein R¹, R² and R³ are all hydrogen and R⁴ is an ethyl substituted with a hydroxyl group, preferably 2-hydroxy ethyl.

3. The method of claim 1, wherein R¹, R² and R³ are all methyl and R⁴ is an ethyl substituted with a hydroxyl group, preferably 2-hydroxy ethyl.

4. The method of claim 1, wherein the organic ammonium carboxylate of formula (I) is a salt of formic acid and monoethanolamine or a salt of acetic acid and monoethanolamine.

5. The method of claim 1, wherein the concentration of the organic ammonium carboxylate of formula (I) in the aqueous solution, is in the range of 1-7% wt-%, preferably in the range of 3-5 wt-% while the freezing point of the composition is kept in the range of −5 to −50° C.

6. The use as defined in claim 1, wherein the aqueous solution of organic ammonium carboxylate of formula (I) contains organic ammonium carboxylate of formula (1) and water in a weight ratio in the range of 1:50-1:15, preferably in the range of 1:33-1:20.

7. The method of claim 1, wherein the aqueous solution of organic ammonium carboxylate of formula (I) contains distilled water only.

8. The method of claim 1, wherein concentration of sodium, potassium, calcium and magnesium is 0 M.

9. The method of claim 1, wherein aqueous organic ammonium carboxylate of formula (I) is additionally admixed with compounds selected from the group comprising glycols, preferably ethylene glycol or propylene glycol, glycerols and viscosity modifying polymers, so that said aqueous composition contains 5 to 97.5 wt-% of water.

10. The method of claim 1, wherein aqueous organic ammonium carboxylate of formula (I) is additionally admixed with auxiliary substances such as additional corrosion inhibitors, biocides, coloring agents, surfactants, and viscosity intensifiers, so that the concentration of auxiliary substances will be in the range of 0.001 to 10 wt-% from the total weight of aqueous solution.

11. The method of claim 1, wherein the organic ammonium carboxylate of formula (1) contains formic acid, acetic acid and monoethanolamine or trimethylmonoethanolamine.

12. The method of claim 1, wherein aqueous organic ammonium carboxylate of formula (I) is used 1-2 I per 1000 kg of crushed stone, preferably 1.2-1.5 I per 1000 kg of crushed stone.

13. The method of claim 1, wherein freezing point of said aqueous solution is from −3° C. to −15° C. preferably from −5° C. to −10° C.

14. The method of claim 1 further including a step wherein aqueous organic ammonium carboxylate of formula (I) is prepared by diluting, in situ, concentrated solution of water and organic ammonium carboxylate of formula (I) with water, in which said concentrated solution the concentration of organic ammonium carboxylate of formula (I) is 50 wt-% or 85 wt-%

15. The method of claim 1, further including a step wherein the aqueous organic ammonium carboxylate of formula (I) is prepared by mixing, in situ, an aqueous solution of 1-5 wt-% of amine with an aqueous solution of 1-5 wt-% of acid

16. A combination of droplets of aqueous solution of organic ammonium carboxylate of formula (I):
[NR¹R²R³R⁴]⁺ₙ [R⁵(COO)]⁻ⁿ, (I),
in which R¹, R², and R³ are selected from the group composing of hydrogen and methyl, R⁴ is a C₁-C₄-alkyl substituted with a hydroxyl group, R⁵ is hydrogen or methyl and n is 1,
and fine material selected from the group composing of sand, crushed stone, minerals and metal powder,
wherein said aqueous solution of organic ammonium carboxylate of formula (I) has been sprayed as a mist onto the surface of said fine material.

17. The combination defined in claim 16 comprising of droplets aqueous solution of organic ammonium carboxylate of formula (I) and fine material, wherein drying of said combination will lead on the one hand to water loss and on the other hand absorbing of water by way of the organic ammonium carboxylate of formula (I) resulting in keeping the surface of said fine material moist.

18. The combination defined in claim 16, comprising droplets of aqueous solution of organic ammonium carboxylate of formula (I) and fine material, wherein organic ammonium carboxylate of formula (I) will absorb water after water content of said aqueous solution have dropped at or below 15 wt-%.

19. The combination defined in claim 16 comprising droplets of aqueous solution of organic ammonium carboxylate of formula (I), and fine material, wherein carboxylate originate from acetic acid, in form of fluid and having BOD (biological oxygen demand) of 0.64 mg of O₂ at 20° C. for liter of said fluid in 5 days.

20. The combination defined in claim 16 comprising droplets of aqueous solution of organic ammonium carboxylate of formula (I), and fine material, wherein carboxylate originate from acetic acid, in form of fluid, and having COD (chemical oxygen demand) of 0.64 mg O₂ at 20° C. for liter of said fluid in 5 days.
